(19) United States
(12) Patent Application Publication
(10) Pub. No.: US 2003/0072676 A1
(43) Pub. Date: Apr. 17, 2003

(54) Radio frequency of electromagnetic information systems and methods for use in extracorporeal blood processing

(76) Inventors: Peter Fletcher-Haynes, Bailey, CO (US); Frank Corbin III, Littleton, CO (US)

Correspondence Address: GAMBRO, INC, PATENT DEPARTMENT, 10810 W COLLINS AVE, LAKEWOOD, CO, 80215, US

(21) Appl. No.: 10/256,852

(22) Filed: Sep. 26, 2002

Related U.S. Application Data
(60) Provisional application No. 60/325,460, filed on Sep. 27, 2001.

(51) Int. Cl. A61L 2/00; A61M 1/14; B01J 19/08

(52) U.S. Cl. 422/23; 422/24; 422/44; 422/186; 422/292; 422/307

(57) ABSTRACT

Containers, methods and systems for treating blood or blood component products; whereby the containers are adapted to contain blood products and the containers have connected thereto respective information/identification chips for use in maintaining information about the blood products which may be contained within the containers and whereby the information/identification chips are adapted for having information written to and read from the information/identification chips including the writing of information to an information/identification chip that the respective blood product was subjected to a pathogen inactivation process.

### INTRODUCTION

The present invention is generally related to medical product information systems and more particularly is directed to the implementation of radio frequency, magnetic and/or electromagnetic communication and data capture devices for product identification and updatable information storage and retrieval in medical device and/or product manufacture, sterilization, storage, and/or use in donor/patient settings, such as in conveying information about extracorporeal blood and/or blood component products, particularly including those which will have been subjected to a pathogen inactivation process.

### BACKGROUND

In the field of collecting and processing blood or blood components from donors for ultimate transfusion to patients, it is desirable to provide donor information and proper identification and categorization of the various blood component products derived therefrom. This has conventionally been performed in the past by printing indicia on a label attached to the face of the blood component container or bag which contains the respective blood or blood component products. However, with respect to a particular blood component which may preferably be subjected to a pathogen inactivation process, including exposure to a pathogen inactivation agent and/or potentially also to irradiation with light, a conventional label on the face of the container or bag may block such light irradiation of the contents thereof resulting in incomplete irradiation and thus a less than successful pathogen inactivation process. Accordingly, it is preferable for the container to be substantially free of labels or like indicia that could block light permeation by irradiation into the blood product container.

Moreover, it is also desirable to have a quick and reliable means for indicating that the blood product within a particular container has or has not been subjected to a pathogen inactivation process such as by exposure to a pathogen inactivation agent and/or irradiation. Also, the indicia used to identify a container will desirably include labeling information or data so that the container and its contents may be tracked and categorized through a computerized inventory control system, which can reduce the risk of misidentification or loss that may be more likely with a manual inventory control system.

Accordingly there is a need for better information and identification devices, systems and/or methods for blood or blood component containers, particularly for pathogen inactivation purposes. Still further, it is desirable to provide for information reading and writing to blood processing containers or devices throughout various portions of a blood processing procedure, including blood component separation and/or pathogen inactivation.

Radio Frequency Identification (hereinafter referred to as “RFID”) is a technology which has recently matured very rapidly. One provider of RFID technology is the Intermec Technology Corporation of Dallas, Tex., USA, which acquired some base technology in electronic chip/microchip applications of RFID from the IBM Corporation, Armonk, N.Y., USA. The RFID technology allows for read/write capability from/onto an RFID chip/microchip using electromagnetic waves, particularly radio frequency (RF) waves, typically at or about 2.45 GHz. Electromagnetic waves of various types including those in the microwave and ultrahigh frequency (UHF) spectra may also be used. A further significant feature of this technology is that the chip/microchip may be powered up by the radio frequency (RF) waves. This means that the information/identification device/chip/microchip may be essentially passive until an RF source is placed in a respective field sufficiently near the device/chip/microchip.

An RFID device/chip/microchip (hereafter referred to as a “chip”) of this sort may then be integrated onto an object. The chip or the object onto which the chip has been integrated may then have a small antenna placed on it which is in energy/data communication contact with the chip. An alternative may be that no actual physical contact of the chip is made to the antenna. The chip may then be mounted in a technique called ‘chip on board.’ This in essence permits the chip to be mounted without the usually associated chip packaging. A typical chip may then be perhaps 2.5 mm square by 1.5 mm deep. The antenna can also be ‘printed’ on the object. The chip may then be soldered or welded onto the antenna. Such a chip assembly can and preferably does withstand gamma radiation, so the complete assembly (the chip device/assembly and the object on which the chip assembly is mounted) can be gamma sterilized. The chip can be programmed either in a locked phase to never erase or it may allow data overwrites. The chip and associated reading/writing devices preferably also have collision detection protocols which allow for one or more chips to be powered in a single RF field and interrogated/read or written to individually or substantially simultaneously while they remain within that same RF field. The distances through which these functionalities may be available are partially a function of transmission power and antenna design. These devices/chips can typically have 1 k bits of storage and have ‘unique’ identifiers. They can also typically be written to with permission 100,000 times or more and read from indefinitely.

### SUMMARY OF THE INVENTION

In accordance with this invention, improvements are provided in information/identification systems and methods for blood product containers and tubing sets. The present invention provides for read/write data storage and retrieval for any blood, blood component or biological liquid product, or any other like material or substance, particularly those subjected to blood processing such as component separation or pathogen inactivation including potentially irradiation with light radiation.

The use of information/identification methods and systems has been known generally in disposable blood tubing sets, as for example, is shown by U.S. Pat. No. 5,769,811 (bar code label on disposable tubing manifold). However, the present concepts involve the incorporation of a type of read and write chip in or onto a blood or blood component container and/or tubing set. Examples include disposition of a read/write chip on a blood component bag or a tubing set cartridge or cassette. With such a read and write device/chip resident on the bag or cartridge, more and better information about the blood component bag or tubing set/cassette or the contents thereof may be written to and stored on the chip at various stages in blood processing, including points prior to, during or even after or as a result of the procedure.

Thus, information such as manufacturing or post-manufacturing inventory data can be written to each chip during many different phases of manufacturing, sterilization, shipping, storage, and/or use in blood processing, and such data need not be written to such chips only once. Moreover, the information written to a chip can be changed as needed. Further, this information can be distinct for each and every bag or unit or cassette, such that each discrete bag and each discrete disposable tubing set can have valuable yet specific information, like container product information and/or volume information or process limitation information specifically written thereon. Further, information can be read from and written to the disposable set/cassette during end-user operation in blood processing such as centrifugation/separation, dialysis or pathogen inactivation. The respective processing machine may also be used to read and/or write to the chip. Thus, user/donor/patient specific data can be written to the chip simply and/or automatically during a run so that donor/patient care or technical operation analysis can later more easily be performed as necessary. If a failure occurs, a donor/patient may be re-started on another processing machine at a recovery point written on the chip, or a technical failure analysis might more easily be performed. In donor situations where the blood product is to be used for later transfusion, donor information (for example blood type or other historical data) may be written to the chip before, during or after the actual donation. Then, any or all of this information can be communicated by/read from the chip before, after or at the point of transfusion to the ultimate recipient of the blood product for improved patient care, inventory control or otherwise.

In accordance with one aspect of this invention, a container may be provided with a chip disposed thereon, the container preferably being photopermeable and thus particularly disposed for pathogen inactivation by irradiation of blood or blood components or other like products. A fluid product may then be provided in such a photopermeable container with a pathogen inactivation agent which may be photoactive and thus the fluid product and pathogen inactivation agent could then be subjected to illumination or irradiation to inactivate any pathogens which may be disposed therein. Non-photoactive pathogen inactivation agents may also be used herein, and such may merely be mixed with the fluid product to be pathogen inactivated. A non-photopermeable container could then be used therewith. The term container refers to a closed or open space, which may be made of rigid or flexible material, e.g., may be a bottle, bag, box, trough, cuvette or fluid cassette (see below). It may be closed or open at the top and/or may have openings at both ends, e.g., the term container may also include flow through devices such as a tube or tubing or a fluid cassette, cuvette or tubing set (see below) to allow for flow-through processing of fluid therein. A container may thus be considered as being exemplified in various embodiments of the invention involving, for example, batch-wise or a flow-through or continuous system. Collection bags, such as those used with the Trima® and Spectra™ apheresis systems of Gambro, Inc., and/or other photopermeable bags suitable for containing fluids may exemplify a preferred embodiment involving batch-wise treatment of a fluid.

The terms light transmissive and photopermeable mean the material of the container is preferably adequately transparent to photoradiation of the proper wavelength for activating the pathogen inactivation agent, if the pathogen inactivation agent is photoactive. In a batch system, the fluid to be treated may be placed in a photopermeable container which may preferably be agitated and exposed to photoradiation for a time sufficient to substantially inactivate any pathogens disposed therein. The photopermeable container is preferably a flexible blood bag made of transparent or semitransparent plastic defining a sealed or sealable blood product chamber, typically having a plurality of conventional access ports for communicating into the blood product bag. The agitating means is preferably a shaker table or member which may preferably be disposed adjacent or in an illumination or irradiation chamber. The pathogen inactivation agent may be added to the container in dry form as a powder, tablet, capsule or pill or in liquid or gaseous form and the container agitated to mix the pathogen inactivation agent with the fluid and to adequately expose all the fluid to the photoradiation to ensure inactivation of any pathogens which may be disposed therein. In an alternative embodiment, the pathogen inactivation agent may be priorly combined with other constituents of an additive solution and such an additive solution containing the pathogen inactivation agent may then be added to the fluid to be treated. It is also contemplated that exposure of the fluid to photoradiation can also occur without agitation of the photopermeable container or that such agitation can occur prior to exposure. The pathogen inactivation agent may be added to the photopermeable container before sterilization of such container or after sterilization.

For both batch and flow through systems, the container walls are preferably made of a plastic material that is substantially transparent to the specific irradiation which may be used for pathogen inactivation purposes. In the preferred irradiation process, a suitable plastic material for use may be a polyolefin or a polyvinyl chloride (PVC). Such materials are readily commercially available. A partially extended seam portion may also be provided, preferably integral with the container but spaced from the interior fluid chamber. The seam portion is preferably made of the same, preferably transparent plastic material from which the rest of the container is made. The information/identification chip label may then be disposed on the container, preferably on or within the plastic seam. As mentioned, the chip label, in turn, allows for reading/writing identifying and/or other information indicia, thereto. It is preferred for the chip label to be particularly disposed on the seam of the container to avoid blocking irradiation of the fluid contents of the container; however, the preferable small size of such a chip would allow for disposition of the chip at any of various locations on the bag. The chip may then have written thereto an indication of exposure of the container and the contents thereof to a particular processing method as for example to a pathogen inactivation procedure which may include the use of irradiation. The chip may alternatively be attached to the container after the irradiation process.

For batch-wise systems, a preferably flexible container is provided in which the walls of the container may be substantially free of opaque indicia to facilitate the irradiation thereof, while the identifying and describing indicia are disposed to reside on an RFID chip which may preferably be positioned on a seam of the container or bag which is spaced from the blood product chamber defined by the container. Information can then be written to the chip at any one or more various points during processing, and then the container can be quickly examined with an RFID reader to determine whether it has been exposed to radiation or otherwise processed for pathogen inactivation through use of the RFID reader to detect the indication of such exposure. The bag seam may also carry an area for receiving handwritten notes or other indicia for added comments and identification by the user.

Additionally, it is preferred that the apparatus for pathogen inactivation, particularly if irradiation is used, may include an RFID reader and/or writer to read from and/or write to the RFID chip before, during or after inactivation and/or irradiation process. Thus, when the bag with an RFID chip is appropriately positioned for pathogen inactivation in an irradiation apparatus, the chip may then be simply and optionally automatically read by an RFID reader and/or written to with an RFID writer. A single RFID device for both reading and writing may be used.

While the container with an RFID chip according to this invention may be used in conjunction with a large variety of different apparatuses for inactivation and/or irradiation, the container is preferably used with an apparatus for irradiating with visible light radiation of a blood component product which may contain pathogens to be inactivated and a pathogen inactivation agent. The irradiation apparatus preferably includes a housing which has a light chamber and one or more light sources positioned within or adjacent the light chamber. A manually removable tray may be disposed within the housing to define a support surface for the blood component container.

In a flow-through system, the container will have a depth (dimension measured in the direction of the radiation from the photoradiation source) sufficient to allow photoradiation to adequately penetrate the container to contact pathogen inactivation agent molecules at all distances from the light source and ensure inactivation of pathogens in the fluid to be decontaminated, and a length (dimension in the direction of fluid flow) sufficient to ensure a sufficient exposure time of the fluid to the photoradiation. The materials for, and the depths and lengths of such containers may be determined by those skilled in the art without undue experimentation following the teachings hereof, and together with the flow rate of fluid through the container, the intensity of the photoradiation and the absorptivities of the fluid components, e.g., plasma, platelets or red blood cells will determine the amount of time the fluid needs to be exposed to photoradiation.

Substances which may be treated and stored using the methods, devices and/or systems of this invention include any substances which may be effectively decontaminated upon exposure to an appropriate pathogen inactivation agent and/or those which may be sufficiently permeable to photoradiation so as to provide sufficient light to achieve pathogen inactivation, or those which can be suspended or dissolved in fluids which have such permeability to photoradiation. Examples of such substances are whole blood and aqueous compositions containing biologically active proteins derived from blood or blood components. Packed red cells, platelets and plasma (fresh or fresh frozen plasma) are exemplary of such blood components. The term “blood product” as used herein includes blood, whole blood, blood components or blood constituents and therapeutic protein compositions containing proteins derived from blood as mentioned above. Fluids containing biologically active proteins other than those derived from blood may also be treated by the methods, devices and/or systems of this invention.

Any means for adding a pathogen inactivation agent or the additive solution containing a pathogen inactivation agent to the fluid to be decontaminated and for placing the fluid in the photopermeable container may be used as known in the art, such means typically including flow conduits, ports, reservoirs, valves, and the like. It may be desirable that the system include means such as pumps or adjustable valves for controlling the flow of the pathogen inactivation agent into the fluid to be decontaminated so that its concentration may be controlled at effective levels as described above. The pathogen inactivation agent can be added to the fluid to be decontaminated in a pre-mixed aqueous solution, e.g., in water or storage buffer solution. Preferably the pathogen inactivation agent is added to the fluid to be decontaminated in aqueous form, but it could also be added as a gas or dry medium in powder, pill, tablet or capsule form.

In one embodiment the fluid is placed in a photopermeable container such as a flexible blood bag, e.g., as used with the apheresis system described in U.S. Pat. No. 5,653,887, inter alia, and preferably agitated while being exposed to photoradiation. Suitable bags include collection bags as described herein. Collection bags used in the COBE Spectra™ processing system or Trima® apheresis system of Gambro, Inc. are examples of bags which may be used here. Alternative shaker tables and like devices are known to the art, e.g. as described in U.S. Pat. No. 4,880,788, for example. The preferred bags are equipped with at least one port for adding fluid thereto. In one embodiment an additive solution containing the pathogen inactivation agent may be added to the fluid-filled bag in liquid form. The bag may then be placed on a shaker table or in an irradiation chamber having a moveable tray (see FIG. 4 as described below) and agitated under photoradiation until substantially all the fluid has been exposed to the photoradiation. Alternatively, the bag may be prepackaged with powdered or fluid pathogen inactivation agent and/or powdered or fluid additive solution constituents contained therein. The fluid to be decontaminated may then be added thereto through the appropriate port.

Decontamination systems as described above may be designed as stand-alone units or may be easily incorporated into existing apparatuses known to the art for separating or treating blood being withdrawn from or administered to a patient. For example, such blood-handling apparatuses include the COBE Spectra™ or TRIMA® apheresis systems, available from Gambro, Inc., Lakewood, Colo., or the apparatuses described in U.S. Pat. No. 5,653,887 and No. 6,200,287, inter alia, of Gambro, Inc., as well as the apheresis systems of other manufacturers. The decontamination system may be inserted just downstream of the point where blood is separated and/or collected or may be disposed just prior to insertion of the blood product into a patient, or at any other point before or after separation of blood constituents. The pathogen inactivation agent may be added to the blood components at any of these points, alone, or in some embodiments along with a storage or additive solution. It is further contemplated that separate irradiation sources and cuvettes could be placed upstream or downstream from collection points for platelets, for plasma and/or for red blood cells. The use of three separate blood decontamination systems would appear to be preferred to placement of a single blood decontamination system upstream of the blood separation vessel of an apheresis system because the lower flow rates in the separate component lines would likely allow greater ease of irradiation. In other embodiments, decontamination systems may be used to process previously collected and stored blood products.

In accordance with this invention, the fluid to be decontaminated is mixed with a pathogen inactivation agent and may then be irradiated with a sufficient amount of photoradiation to activate the pathogen inactivation agent to react with pathogens in the fluid such that such pathogens in the fluid are inactivated. The amount of photoradiation reaching the pathogens in the fluid is controlled by selecting an appropriate photoradiation source, an appropriate distance of the photoradiation source from the fluid to be decontaminated, and appropriate photopermeable material for the container for the fluid, an appropriate depth to allow appropriate penetration of the photoradiation into the container, and optional photoradiation enhancers such as one or more additional photoradiation sources, preferably on the opposite side of the container from the first, or reflectors to reflect light from the radiation source back into the container. If a flow through system is used, appropriate flow rates for the fluid in and through the container and an appropriate container length to allow sufficient time for inactivation of any pathogens present are also selected. Temperature monitors and controllers may also be required to keep the fluid at optimal temperature.

For batch systems, it is preferred to place the fluid to be decontaminated along with the pathogen inactivation agent in bags which are photopermeable or at least sufficiently photopermeable to allow sufficient radiation to reach their contents to activate the pathogen inactivation agent. A sufficient quantity of pathogen inactivation agent along with any storage or additive solution is added to each bag to provide inactivation, preferably to provide a pathogen inactivation agent concentration of at least about 10 μM, and the bag may be agitated while irradiating, preferably at about 1 to about 200 J/cm² for a period of between about 1 to about 60 minutes to ensure exposure of substantially all the fluid to light radiation. Visible or ultraviolet or a combination of visible light and ultraviolet light may be used. The fluid to be decontaminated may also contain additives or anticoagulant solutions and the blood product or blood components may then be stored in such solutions. Examples of methods and materials useful with the present invention are disclosed in U.S. Pat. No. 6,277,337 and No. 6,258,577, inter alia.

These and other features of the present invention will be made manifest by the following detailed description and the attached drawings which are intended to be read in conjunction with each other as set forth below.

### BRIEF DESCRIPTION OF THE DRAWINGS

In the drawings:

FIG. 1 is a plan view of a blood or blood component container having an information chip disposed thereon;

FIG. 2A is a partial cross-sectional view taken along line 2A/B-2A/B of FIG. 1;

FIG. 2B is an alternative partial cross-sectional view also taken along line 2A/B-2A/B of FIG. 1;

FIG. 3 is a schematic view of a system of blood or blood component and/or biological fluid containers having one or more respective information chips disposed thereon;

FIG. 4 is an isometric view of an apparatus in which a blood or blood component container may be disposed for a pathogen inactivation procedure;

FIG. 5 is a plan view of a blood or blood component container in use with an optional hand-held information chip reader and/or writer; and

FIG. 6 is a blood processing tubing set with a fluid cassette having an information chip disposed thereon.

### DETAILED DESCRIPTION OF THE INVENTION

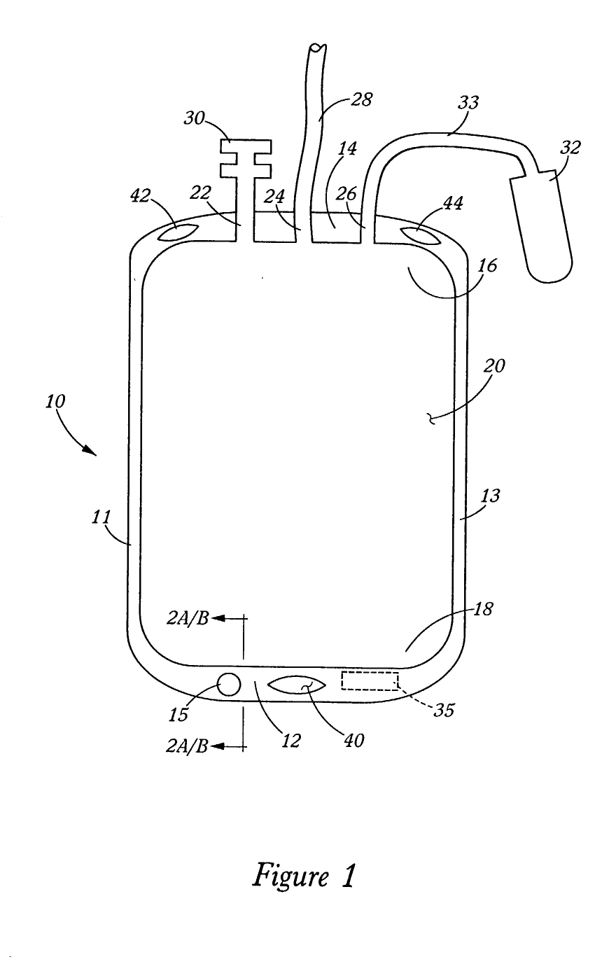

Shown in FIG. 1 is a container 10 for a fluid or blood product such as blood or blood components which may be or which may have been subjected to extracorporeal processing such as preferably a pathogen inactivation process which may include exposure to a pathogen inactivation agent and/or to light irradiation. An electromagnetically operable information chip 15 such as the preferred read/write radio frequency identification (RFID) chip 15, as shown in FIG. 1, is attached to container 10. Container 10 may take various forms but is preferably a flexible bag and may be made in accordance with substantially conventional methods being formed preferably from two peripherally heat sealed sheets of a preferably light transmissive (also known as photopermeable, inter alia) or substantially transparent plastic such as a polyolefin or polyvinyl chloride (PVC) material peripherally sealed together by a radio frequency (RF) heat seal or weld or other type of seal or seam as known and shown by boundary seams 11, 12, 13 and 14. These seams preferably form a continuous seal or seam about the circumference of the bag 10 (with the preferred exception of one or more access ports, see below). Seams 11, 12, 13, and 14 thereby define an internal fluid chamber 20 within bag 10. Note, the seal or seals used to define chamber 20 and create seams 11, 12, 13 and 14 may, but need not, extend to the extreme outer edges of seams 11, 12, 13 and 14 as shown here. Non-sealed portions of seams 11, 12, 13 and 14 may then result, though still maintaining a peripherally sealed chamber 20 as defined above.

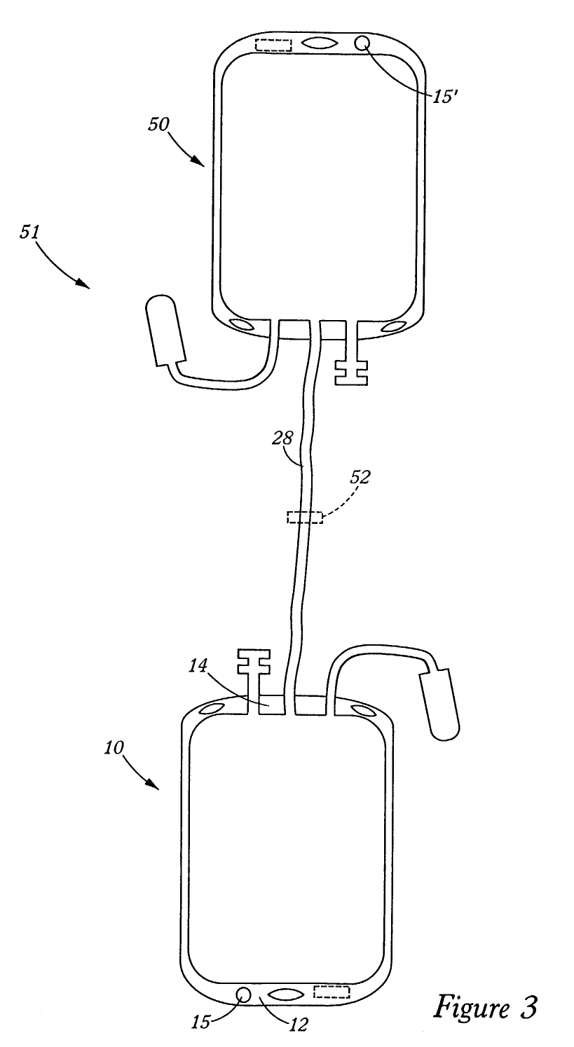

Container 10 also includes respective opposing container ends 16 and 18, with at least one and preferably, as shown, three access ports 22, 24, 26 being defined at one end 16 thereof. These ports are preferably formed in a substantially conventional manner. Central access port 24 may be, as shown, connected to a length of flexible tubing 28, which may be pre-connected or otherwise connectable or sterile dockable to another container or bag (not shown in FIG. 1, but see FIG. 3 as described below), or may terminate in a conventional spike or sharp/needle connector (not shown) for post-manufacture connection to a discrete fluid or blood product source. The other two access ports may also be connected to discrete tubing lines (one shown, see below) or may have other devices connected thereto. For example, port 22 may, as shown, be connected to a preferably semi-rigid, tubular port structure or protector 30 which may contain a spike puncturable diaphragm (not shown) to close off the port 22 in conventional manner, yet allow for subsequent access as may be desired. Another port connection example may be the connection of a sample bulb 32, as shown, or a sample bag (not shown) connected to port 26. A tubing line 33 may be used for this connection. The relative locations of these ports is shown and described in a preferred fashion for convenience, but may take other forms and dispositions about the periphery of container 10, as may otherwise be desired.

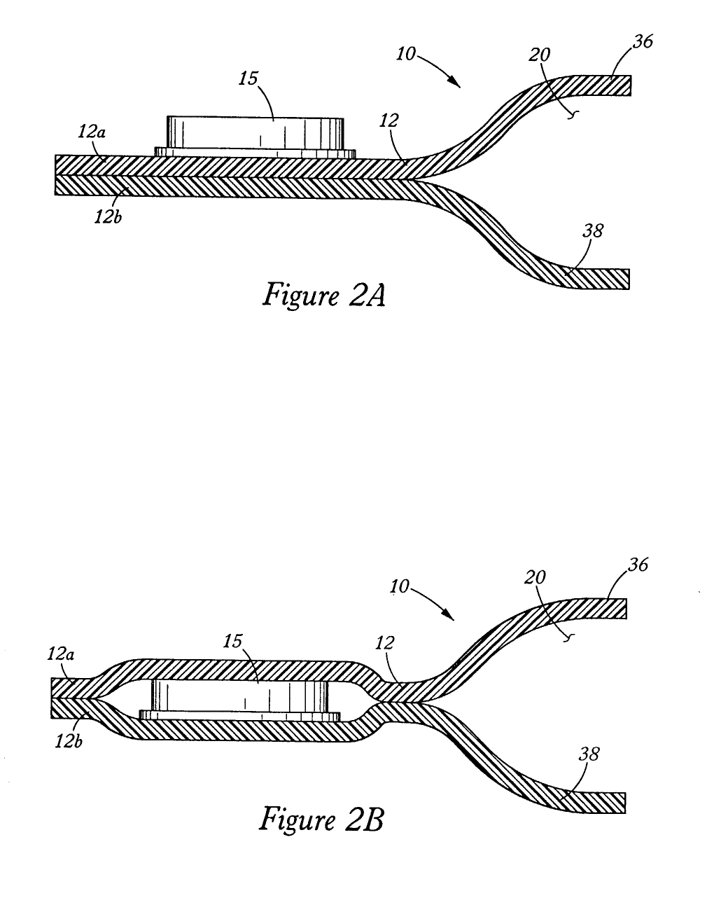

At the opposite end 18 of container 10, seam 12 is defined as described above, adjacent and abutting the internal fluid chamber 20 of container 10, as this chamber is defined within the peripheral seal established by boundary seams 11, 12, 13 and 14. As mentioned, an information chip 15 (interchangeably also referred to throughout this specification as an RFID chip 15) is attached or connected to bag 10 and is preferably disposed in or on seam 12. As such, the information or RFID chip 15 may be affixed to the external surface of seam 12, as shown in FIG. 2A, or may be disposed between the plastic sheets and thus welded in place therebetween as shown in FIG. 2B. Note, in either case, the chip 15 may be affixed to the corresponding side portions 12a and/or 12b of seam 12, as by gluing, solvent bonding or using other adhesive means or it could be melt-welded into or against the plastic, though in the FIG. 2B embodiment the chip 15 may be secured in place without such adhesive means and remain appropriately disposed in place by the action of the seam 12 welds therearournd. Seam 12 may further be made large enough from the welded plastic sheets to also have disposed thereon or therein an optional printed or printable label or card 35 (shown in dashed lines in FIG. 1). Any of seams 11, 12, 13 or 14 may be adapted to have the RFID chip 15 and/or the label 35 disposed therein or thereon. Printed label 35 may be opaque, translucent or substantially transparent, or may have various indicia printed or printable thereon, including, for example, printed words, symbols or a code such as a bar code which is readable by a conventional bar code reader. Note, the chip 15 and/or the optional label 35 may be disposed on or be sealed within the seam 12 so long as the RF (or bar code) reader/writer may still be operably active through the preferably photopermeable and/or transparent plastic of the plastic sheets which may form seam 12. Chip 15, label 35 or a further paper label (not shown) may be adhered to the outer and/or reverse surface of seam 12 (or any other seam 11, 13 or 14) as well. A label 35 or the like permits the user to write notes or indicating indicia thereon with a pen or pencil or like writing implement.

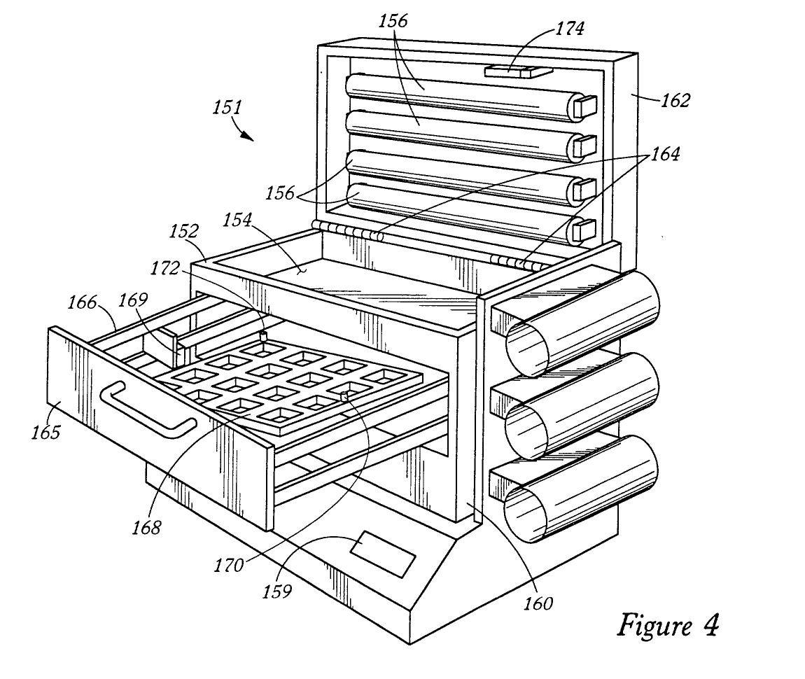

Other features may also be defined in or on the seams 11, 12, 13 or 14. For example, as shown in FIG. 1, an aperture 40 may be cut or punched or otherwise formed in seam 12, preferably being defined through the two joined plastic layers of seam 12. Seam 14 may similarly have defined therein a pair of apertures or holes 42, 44, as might seams 11 and/or 13 (not shown). Any of these holes 40, 42 and/or 44 may be positioned to be received on hooks (not shown) for hanging purposes during fluid transfer (e.g., by gravity or pump-assisted drainage; see the description relative to the schematic of FIG. 3, below) and or for facilitating and/or securing the disposition of the bag 10 in a pathogen inactivation apparatus (see FIG. 4). These apertures 40, 42 and 44 may be made in container 10 to properly position container 10 in a preferred manner for pathogen inactivation which may include light irradiation of the contents thereof by the pathogen inactivation apparatus (see description relative to FIG. 4).

Container 10, particularly the container side walls 36, 38 (see FIGS. 2A and 2B) defining the interior chamber 20, is preferably free of any opaque indicia, with all of the indicia being disposed on the seams 11, 12, 13 and/or 14, and preferably having the indicia electromagnetically disposed in the RFID chip 15. Thus, there would preferably be presented no opaque interference with the light irradiation of the contents of the container 10. Alternatively, a label (not shown) or an RFID chip 15 could be attached to a side wall 36 or 38 (and not necessarily on a seam 11, 12, 13 or 14), preferably after any light radiation processing (if used) for one or more of the information purposes described hereinbelow. Such a post-attached label could then be more opaque or translucent (though it could also be substantially transparent). Or, an alternative, substantially transparent, label may be disposed on one of side walls 36, 38 before (or after) exposure to light irradiation with the preferably subsequent (but acceptably prior) application of the printed matter (words, symbols, bar codes or the like) thereto. Note also, though less preferred, the RFID chip may be positioned on a respective sidewall 36 or 38 prior to and remain there during an irradiation procedure. Optimization of such a procedure may then be obtained using a movement or agitation apparatus to mix and/or move the fluid contents of the container 10 and thus avoid the potential for incomplete exposure of the fluid and photosensitizing agent to the light source.

Note also that as introduced above, a chip 15 may be added/attached at any of various points in a procedure and as will be described further, a chip 15 may be moveable such that in one example, it may be removable from a container 10 and used for various purposes including remote reading and/or writing of information from or to the chip. Removing a chip 15 may also be used in connection with transporting or transposing one chip 15 from one container or like device to another device or container (see below).

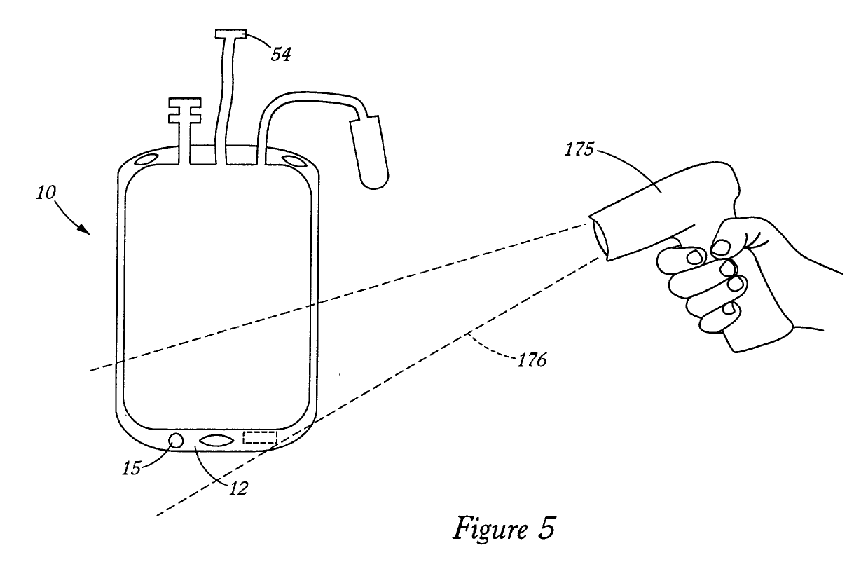

In FIG. 3, a combination or system 51 of containers or bags 10 and 50 is shown. Thus, a bag 10 as from FIG. 1 may be connected in fluid communication relationship with another container or bag 50 as shown through tubing 28 such that the contents of one or the other container or bag 10 or 50 may be communicated or transferred to the other container or bag 50 or 10. In one embodiment, a bag 50 is a collection bag for the original collection of blood or blood components (as from a donor (not shown) and/or from separation through centrifugation (also not shown), e.g.), which may then be transferable to a light permeable and thus irradiatable bag 10 for further processing as described herein below. Or, container 50 may be a container for a pathogen inactivation agent (not separately shown) which may be transferable into the blood product bag 10 to be mixed with the blood or blood component product which is to undergo the pathogen inactivation process. In either case, the containers/bags may be pre-connected during manufacture or sterile docked or spike connected or otherwise later connected at a connection such as connection point 52 (shown in dashed lines) as may be understood in the art. Disconnection after fluid or blood product transfer may also be effected as understood, as for example, using an RF welding device which may also be used to provide for the tearing or cutting of the tubing line 28 at a weld line 54 (see FIG. 5 as described below).

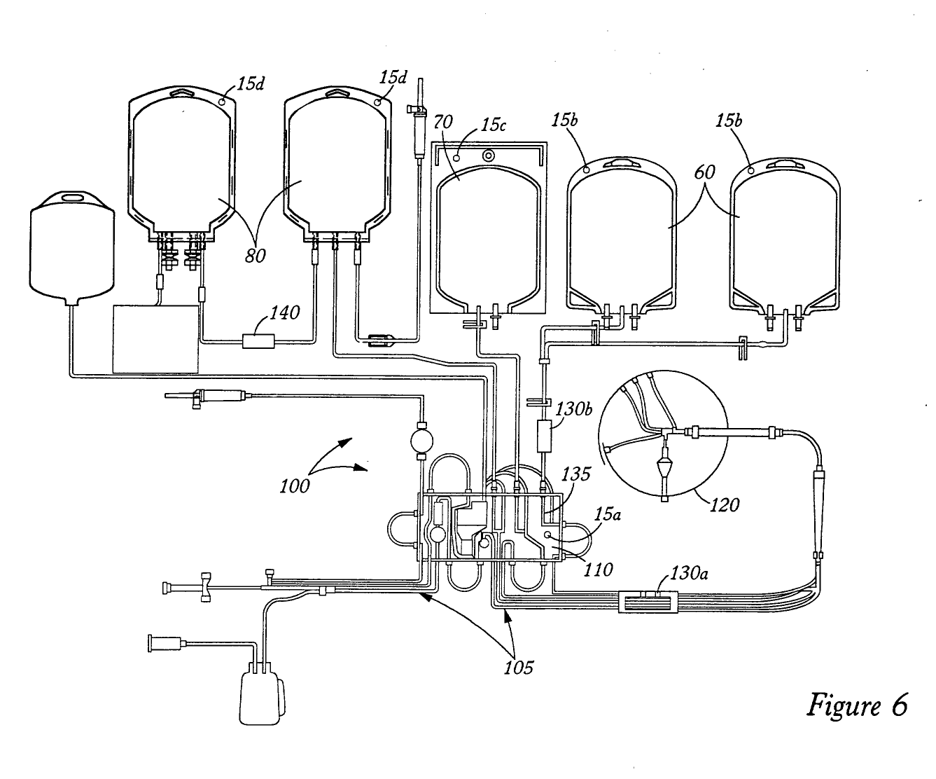

FIG. 3 depicts an embodiment of this invention in which blood bags or other containers used in blood processing and/or storage may be prepackaged in one preferred embodiment of this invention to contain a pathogen inactivation agent in either dry or aqueous/fluid form. The embodiment depicted in this FIG. 3 may be used with the collected blood component bag 10 of FIG. 1 as well as and/or alternatively in the system 100 depicted in FIG. 6 (see below). If used, additive constituents optionally useable for storage of blood components may also be prepackaged either separate from or together with the pathogen inactivation agent. It is further understood that the pathogen inactivation agent and blood component additives that may be prepackaged within the bags may be in a gas or a dry powder form, a pill, capsule, tablet form, liquid form, or in various combinations thereof. In describing this invention, the term dry solid or dry form envisions the components being in a loose powdered state or in a solid state such as a pill, capsule, tablet, or any equivalent thereof known to one skilled in the art. The term biological fluid is intended to encompass both biologically-created fluids (such as blood and its components) as well as biologically compatible additives (such as a pathogen inactivation agent and/or storage solutions, inter alia).

As shown in FIG. 3, a first bag 10 and a second bag 50 are connected together by flexible tubing 28 to form a system 51. The first and second bags 10 and 50 could also have one or more other containers (not specifically shown) located between or otherwise connected to one or the other of the two bags. Such a container could be another bag, a flask, a reservoir, a small cylinder or any similar container known in the art, and such container or the tubing 28 itself, could contain certain forms of prepackaged components, in a manner similar to that of the two bags 10 and 50.

In a first alternative embodiment, the pathogen inactivation agent and/or an optional blood additive and/or physiological saline may be prepackaged in the second bag 50. The pathogen inactivation agent and/or optional additive components may be in a dry solid, a gas, or in a liquid form. If a dry form is used, a solution or preferably saline solution may be added to the bag 50 through a port. Other additives may be included or added at various points, e.g., a tertiary bag (not shown) may also be prepackaged and the contents thereof later added to the second bag 50. Upon or prior to or during addition of the separated blood component to the first bag 10, the fluid mixture containing a pathogen inactivation agent and/or optionally an additive solution may then be moved via the flexible tubing 28 from bag 50 into the first bag 10. The first bag 10 may then be disconnected from the second bag 50 for pathogen inactivation of the blood component, including mixing and/or irradiation. It should be noted however, that either the first bag or the second bag could be used for inactivation as long as the inactivation (which may optionally include mixing and/or irradiation) is performed preferably after the addition of the pathogen inactivation agent.

In an alternative embodiment contemplated by this invention, the first bag 10 may contain a pathogen inactivation agent with or without a possible additive solution and the second bag 50 may represent the collected blood component bag from which the blood component is moved into the first bag 10 (or vice versa) for inactivation (which may optionally include mixing and/or irradiation). The use of a frangible connector (not shown) between the first bag 10 and the second container 50 is further envisioned for use with this invention. The frangible connector would then be manually snapped to allow fluid or blood product in one respective bag 10, 50 to reach the other fluid in the other corresponding bag 50, 10 when desired.

In another embodiment, two separate mixtures may be disposed in bags 10 and 50 for separate sterilization. Then, before use and after sterilization the respective contents of bags 10 and 50 may be mixed preferably in one bag, e.g., bag 10, to form a third aqueous mixture (although the contents of both bags could also be mixed in the bag 50). The pathogen inactivation agent mixture (the third mixture) could then be combined with the collected blood component, for example, the platelets collected using the apheresis apparatus of FIG. 6 (see below). By way of example, the contents of one platelet collection bag 60 (FIG. 6) which could be considered as representing bag 50 from FIG. 3, may be added to the pathogen inactivation agent in a bag 10 as in FIG. 3. The fluid or blood component may then be mixed and/or irradiated, as will be further described below. It is also understood that the pathogen inactivation agent mixture can be added to bag 60 (FIG. 6) (which may then represent bag 10 of FIG. 3) and the fluid or blood component can be irradiated in that collection bag 60. The preference is that the fluid or blood component to be decontaminated by light irradiation be combined with the pathogen inactivation agent and any optional additive constituents and may then be disposed in the bag which is permeable to photoradiation for optional photo-inactivation.

Turning to FIG. 4, an apparatus 151 which may be used in a preferred irradiation procedure of the container 10 of FIGS. 1 and 3 is shown. Apparatus 151 preferably includes a housing 152 which has an internal chamber 154, with respective banks of lights 156 (only one bank of lights is shown in FIG. 4) on opposed sides of chamber 154 in the housing 152. Housing 152 may comprise a pair of housing sections 160, 162 which may be connected together on one side with hinges 164 so that the housing sections may be movable in hinged relation between an open position as shown in FIG. 4 and a closed position which is not shown. The irradiation preferably takes place in the closed position. Each of sections, base 160 and lid 162, preferably has disposed therein a respective one of the banks of lights, e.g., bank 156 in lid 162 (only the one bank being shown in FIG. 4), plus the circuitry (not shown) for operating them. The circuitry is ultimately connected to a control panel 159 disposed on the apparatus 151. A movable tray 165 is preferably provided to be moved into and be operably disposed within the housing sections of apparatus 151 when the apparatus 151 is in its closed position. Thus, the apparatus can be opened, and the tray 165 may be caused to slide outward to the position shown and then simply replaced, the tray being appropriately positioned so that both sides of it may be irradiated by the light banks, e.g., bank 156 and the unshown bank in base 160. Tray 165 is preferably defined by a frame 166 and a bottom member 168 (which may preferably be a wire or like mesh or transparent plastic to allow maximum light transmissivity therethrough). In a preferred embodiment bottom member 168 is also moveable by a small motor 169 in order to oscillate, nutate or shuttle (swash and/or wobble) and thereby agitate and/or mix the contents of the bag 10 during the irradiation process. Thus, a flexible container 10 such as that shown in FIGS. 1 and 3 may be placed on the bottom member 168 of the tray 165 to form a blood product layer which is spread out to the degree permitted by the flexible container 10 (not shown in FIG. 4). Then, tray 165 may be placed into irradiation apparatus 151 and the housing sections 160, 162 may be closed by a pivotable hinged motion into their closed configuration. The light banks may then be actuated for a predetermined time as set by means of the apparatus controls or panel 159, e.g. with the bottom member 168 preferably continuously agitating the contents of bag 10. Tray 165 may have one or more pins, e.g. pin 170 and pin 172, which may be positioned to pass through holes 40, 42, and/or 44 of container 10 to properly position the container on bottom 168 within apparatus 151. Thus, with such proper positioning, chamber 20 of bag 10 may be properly positioned for optimum exposure to light from the respective banks of lights, e.g., bank 156. RFID chip 15 (not shown in FIG. 4) may also be positioned in apparatus 151 for reading and/or writing by an RFID reader/writer 174 which may be, as shown, disposed in housing section 162 or in section 160 (not shown). Such readings and/or writings may then be made on command (through control panel 159 or the like) or automatically as may be desired.

Whether disposed in apparatus 151 or disposed externally thereof, as by a hand-held or like reader/writer 175 (see FIG. 5), data may be read from and/or written to the RFID chip 15 through an electronic and/or a software driven system which may be separate from or included in or connected or connectable to an apparatus 151. The RFID reader/writer 174 (or a hand-held or like reader/writer 175, FIG. 5) can cross-check with other data or criteria to be sure that the contents of bag 10 are being properly treated, and ensure that the identity of the contents of bag 10 has been verified. Appropriate information from the RFID data can be read out or printed out for the users as may be desired e.g., before, during or after processing or other blood product use. The system may be disabled if the RFID data is not that which was expected by the program.

The fluid containing the pathogen inactivation agent is preferably exposed to photoradiation of the appropriate wavelength to activate the pathogen inactivation agent, using an amount of photoradiation sufficient to activate the pathogen inactivation agent as described above, but preferably less than that which would cause non-specific damage to the biological components or substantially interfere with biological activity of other proteins present in the fluid. The wavelength used will depend on the pathogen inactivation agent selected, as is known to the art or readily determinable without undue experimentation following the teachings hereof. Preferably the light source is a fluorescent or luminescent source providing light of about 300 nm to about 700 nm, and more preferably about 340 nm to about 650 nm of radiation. Wavelengths in the visible to ultraviolet range may be useful in this invention. The light source or sources may provide light in the visible range, light in the ultraviolet range, or a mixture of light in the visible and ultraviolet ranges.

The activated pathogen inactivation agent may then be capable of inactivating the pathogens present, such as by interfering to prevent their replication. Specificity of action of the pathogen inactivation agent may be conferred by the close proximity of the pathogen inactivation agent to the nucleic acid of the pathogen or microorganism and this may result from binding of the pathogen inactivation agent to the nucleic acid. The term nucleic acid includes ribonucleic acid (RNA) and deoxyribonucleic acid (DNA). Other pathogen inactivation agents may act by binding to cell membranes or by other mechanisms. The pathogen inactivation agent may also be targeted to the pathogen or microorganism to be inactivated by covalently coupling to an antibody, preferably a specific monoclonal antibody to the pathogen or microorganism. The pathogen inactivation agent may be photoactive or not, and if not, then a pathogen inactivation device, such as apparatus 151 (FIG. 4) may be used merely to mix a pathogen inactivation agent with the blood component product via a moveable member 168.

In a preferred embodiment, after the inactivation has taken place, the reader/writer 174 in device 151 (FIG. 4) or the external reader/writer 175 (FIG. 5) may write to the RFID chip 15 that the pathogen inactivation process has been completed for the contents of this particular bag 10. Then, the container 10 may be removed from the apparatus 151 and may then optionally be replaced with another container 10 containing another blood product for a subsequent inactivation process. If an external reader/writer 175 (see FIG. 5) is used as an alternative to an internal reader/writer 174, then the writing to the RFID chip 15 that the pathogen inactivation process has been completed, may preferably be accomplished after sliding the tray 165 out of apparatus 151 and/or removing the bag 10 therefrom.

Note, other information may also be written to the chip 15 at any time, for example, if desired, information such as donor-related data and/or data concerning the blood product itself, the blood type, the type, time and/or date of separation process used, additives added, anticoagulant used, volume(s) collected, time held before, during or after pathogen inactivation, type, time and/or date of inactivation (including, e.g., pathogen inactivation agent used, whether photoinactivation is also used or necessary for the particular agent) and other indicia of process conditions. Again, these types of information may be written to the chip before, during or after the particular processing episode, e.g., donor information may be written to the chip before, during or after the donation event. Confirmation protocols may also be activated by the types of data written to a chip 15, e.g., a particular type of blood component may require or be designated for use only with a particular type or volume or concentration of pathogen inactivation agent and/or additive and/or a particular type of photoinactivation. The inactivation or irradiation chamber (or the blood bank system) may then refuse a blood product until or unless certain prior steps are completed, e.g., addition of a proper inactivation agent (type, volume and/or concentration) for a particular blood component. Similarly, the chip 15 can be used to communicate to the processing equipment such that the process can be varied to obtain optimized results. For instance, if the volume of the particular blood product is varied a certain amount, perhaps this information can be written to the chip 15 and then communicated to the processing apparatus, e.g., apparatus 151, which may then be able to vary the irradiation or illumination intensity and/or time period accordingly.

Other information relative to other points of blood product processing or handling may also be written to a chip 15. For example, post-separation, post-inactivation (if used) procedures such as freezing plasma or red blood cell products might also involve information which could be recorded to a chip 15 before, during or after freezing. Thus, a chip 15 could, in one alternative embodiment, be disposed on a bag 10 and subjected to freezing therewith. Other alternatives could include removing a chip 15 prior to freezing and/or placing (or replacing) a chip 15 on a respective bag 10 after the bag 10 is removed from the freezing process and perhaps after the thawing process is completed.

Thus, an inactivation apparatus 151 and a container 10 for use therein is disclosed, having significant advantages based on monitoring of the identity of and information about the blood product being subjected to a pathogen inactivation process, for added reliability of identification of and information about the blood product, and to further provide the confidence that blood products will not become mixed up or processed wrongly. Additionally, the RFID chip provides information for determining whether the contents of a container have been inactivated as a further cross-check against undesired double inactivation or failure to inactivate. Thus, pertinent data may be communicated to or for the ultimate user or recipient (e.g., transfusion patient) of the blood product by the chip. Moreover, while all desired identifying indicia may be applied to the bag via the RFID chip, the chamber that holds the blood product is then preferably not shadowed and shielded from irradiation by any indicia carried on the container wall that surrounds the chamber, although even if partially shielded, mixing can also be used in an embodiment for achieving optimum irradiation.

Note, the readers/writers (e.g., 174, 175) may be active in near field and/or far field (see, e.g., field 176 in FIG. 5) and may use RF or microwave or ultra high frequency spectra or may be effective in other magnetic or electromagnetic wave spectra. In a typical combination, RF chips and readers/writers may be operated with anti-collision protocol reading to read as many as 50 chips or more per second in a reading zone or range of ten meters, for example. Such systems are available from the Intermec Technologies Corporation, Dallas Tex., U.S.A. Such chips may also be written to and have data written thereto over 100,000 times, and writing may be locked at certain points so that such data may not be overwritten. Thus, in a final stage of processing, the data may be locked to reduce risk of misidentifying a product, e.g., a pathogen inactivated product. Patient transfusion quality is thus enhanced.

Additionally, information and/or data may be read from or written to a chip 15 through a reader/writer 174 and/or 175 to and/or from a computer (not shown) at any of various points of fluid component processing or handling. Such computer use could include any of many forms including product tracking or inventory control, as well as alternatively related to donor information control, process quality control, and/or end user or patient usage, particularly in data retrieval for appropriate product-patient matching in transfusion settings. In any such usages, the data transmissions to and/or from a computer (not shown) from and/or to a chip 15 may also be direct as in having a reader/writer 174 and/or 175 directly connected to a computer, and/or the transmissions may be indirect such that the reader/writer 174 and/or 175 may not be directly connected to a computer. Instead, the data may be temporarily stored otherwise or may be directed to other devices, e.g., such as to an apparatus 151 or hand-held device 175 for use therein or thereby and potentially also for later transfer or transmission to a computer. Thus, in an embodiment, information disposed on a chip 15 may be read therefrom into a computer (not shown) prior to a processing event such as pathogen inactivation. The information may then be used by the computer in verifying and/or assisting and perhaps varying the procedure (e.g., varying irradiation intensities and/or times based upon the chip information), and then (before, during or after the procedure), further information can be written from the computer or other device to the chip about the procedure (e.g., procedure completion) and/or data in the computer can be updated as to such completion of the procedure. Verification of such completion of a procedure may then be made subsequently at the point of patient transfusion either through computer matching, or by subsequent data reading with a discrete reader/writer 175.

Furthermore, an RFID chip 15 may be placed on other component parts of a blood processing assembly, such as the tubing and bag assembly 100 shown in FIG. 6. More particularly, a chip, here labeled 15a, may preferably be placed on a central or other preferably sturdy element such as a cartridge or cassette 110 of the processing assembly 100 as shown. A processing assembly including such a cassette 110 is described in more detail in U.S. Pat. No. 5,653,887; No. 6,200,287; inter alia and PCT publication WO 01/24848; inter alia, for example, the disclosures of which are hereby incorporated herein by this reference thereto. In this alternative embodiment, an RFID chip 15a may serve to identify the blood tubing and bag set 100 which is preferably adapted to provide for centrifugal separation in separation vessel 120, of blood into component parts such as plasma, platelets and/or red blood cells (RBCs). These components may then be collected in respective sets of containers 60, 70 and 80 as shown in FIG. 6. As described in further detail in the preferred embodiment and as set forth in the above-listed U.S. patents, platelets may be collected from vessel 120 into container(s) 60, plasma into container 70 and RBCs into container(s) 80. Each or some of these containers 60, 70, and 80 may then also have respective RFID identification chips 15b, 15c and 15d as shown. These containers 60, 70 and/or 80 may then be subjected to pathogen inactivation procedures as described above.

FIG. 6 shows a blood processing apparatus for use in an apheresis system for separating and collecting blood components which may also include processing by or provide for separate further processing by exposure to a pathogen inactivation agent. Whole blood may be withdrawn from a donor/patient (not shown) and provided to an apheresis system and/or a blood processing apparatus/tubing set 100 and moved to the centrifuge vessel 120 where the blood may be separated into various component types. At least one of these blood components may then be removed from the vessel 120 and respective blood components may then be collected and provided for subsequent use by another or may undergo a therapeutic treatment and be returned to the donor/patient. A processing machine which may be used with a tubing set 100 or a like tubing set (not shown) may be such as the COBE Spectra™ and/or Trima® apheresis machines (not shown, see examples of those described in the above-listed patents), or may be such as those processing machines of other manufacturers such as Baxter (the Amicus, CS-3000 and Autophoresis-C), Haemonetics (MCS and MCS+) and/or Fresenius (AS-104 and Com-Tec), machines, inter alia.

In the shown embodiment of the blood component processing apparatus 100 (FIG. 6), blood is withdrawn from the donor/patient and directed through an extracorporeal tubing circuit 105 and a blood-processing vessel 120 which preferably define a closed and sterile system. The blood component processing apparatus 100 may be connected to one or more pumps (not shown) at least one of which may cause blood to flow from the donor/patient through the extracorporeal tubing circuit 105 and into a rotating blood processing vessel 120. The blood within the blood processing vessel 120 is separated into various blood component types, and these component types (platelets, plasma, red blood cells) can then be continually removed from the blood processing vessel 120. Blood components which are not being retained for collection or for therapeutic treatment (e.g., red blood cells, white blood cells, plasma and/or platelets) are also removed from the blood processing vessel 120 and returned to the donor/patient via the extracorporeal tubing circuit 105. Operation of the blood component separation device may preferably be controlled by one or more computer processors included therein.

Extracorporeal tubing circuit 105 may include a cassette assembly 110 and a number of tubing assemblies interconnected therewith as shown in FIG. 6. The platelet collection assembly 60, plasma collection assembly 70 and red blood cell collection assembly 80 are also interconnected with cassette assembly 110. Cassette assembly 110 preferably includes front and back molded plastic plates (which may also be known as respective side walls) that are preferably hot-welded together to define a cassette member preferably having integral fluid passageways. Thus, cassette 110 may also be a container according to the present invention, and the welded and/or non-fluid-channel portions of the side walls may also be seams. Note, tubing set apparatus 100 may also be considered a container in either view that it has both flow-through and receptacle container parts.

The embodiment of FIG. 6 also allows for depictions of alternative flow-through embodiments to the stand-alone, batch-wise version of the decontamination assembly of this invention. Utilizing a flow-through concept, the blood product (which may be recently collected blood, stored blood or a blood component, for example, separated platelets which are moving from vessel 120 to a bag 60 may be flowed through a decontamination cuvette 130a which may be prior to cassette 110 or may be in/on cassette 110 (as for example in or form part of flow channel 135 in cassette 110, not shown) or may otherwise be separate therefrom after cassette 110 as depicted by cuvette 130b in FIG. 6. A discrete example of a disjoined cuvette 140 is also shown in FIG. 6 and is described below relative to the alternative product collection bag(s) 80.

In the first example relative to either cuvette 130a, 130b or the flow channel 135, a pathogen inactivation agent reservoir (not shown) may be connected to the pathogen inactivation tubing line upstream from the decontamination cuvette 130a, 130b or channel 135. The decontamination cuvette 130a or 130b, or channel 135 would then be connected to or disposed in or adjacent to a photoradiation source (not shown), preferably disposed on an apheresis machine (not shown). The photoradiation source could then be disposed on the face of the processing machine, in either case, and particularly could be disposed under the channel 135, for example. A decontaminated blood product tubing line then leads from the decontamination cuvette 130a or 130b (whether in (see channel 135) or disjoined from cassette 110) and ultimately goes to the decontaminated blood product collection bag 60, e.g. This line in FIG. 6 first leads from a cuvette 130a to the cassette 110 and then to the bag 60, or however, an alternative disjoined cuvette 130b could be disposed downstream of cassette 110 and may then lead directly to the collection bag(s) 60.

In operation, the blood product (such as platelets, here) is conducted from the separation vessel 120 into a blood product tubing line where it may be joined by the pathogen inactivation agent and optionally also with an additive solution from a pathogen inactivation agent reservoir (not shown). The blood product in decontamination cuvette 130a, 130b or flow through channel 135 may be irradiated by photoradiation from the photoradiation source (not shown). The photoradiation source may comprise one, two or more actual lights. Decontaminated blood product may then exit decontamination cuvette 130a or 130b or flow channel 135 and be collected in a decontaminated blood product collection bag 60. Flow rates, illumination wavelengths and temperatures may be controlled by the blood processing machine (not separately shown) to achieve optimal operating conditions.

In an example including a cuvette 140, wherein the cuvette 140 is disposed in a tubing line connecting two bags 80, the pathogen inactivation agent could be added by various methods including having resided in either of the initial bags 80 or having been added to the primary bag 80 using a spike or like connector (as shown). Then, the separated product could be mixed therewith in a fashion such as those alternatives described relative to FIG. 3, inter alia. Flow of a separated product mixed with a pathogen inactivation agent could then proceed from one bag 80 to the other through cuvette 140 which could then be exposed to irradiation to activate the pathogen inactivation agent to inactivate pathogens. As shown and described, this could take place in a flow-through fashion.

As set forth above, data may be written to the chip(s) 15, 15′, 15a, 15b, 15c or 15d at any time during any process or procedure and may include data elements beyond notification of completion of a pathogen inactivation process. Examples include process conditions existing before, during and/or after a procedure such as volumes processed and/or collected, concentrations or yields predicted and/or collected, other process variables such as additives or anticoagulants (if used), types of procedures, speeds of pumps or centrifuges, flow rates, even process start/stop information, alarms experienced and time of procedure information; donor data including blood type, hematocrit, hemoglobin, height, weight, gender, replacement solutions (if used), as well as pathogen inactivation variables including, for example, the type of pathogen inactivation agent used and/or whether and what type of light irradiation was used.

In the present invention with or without pathogen inactivation, the chip or chips 15, 15′, 15a, 15b, 15c and/or 15d may be placed on and/or transferred between any or every medical disposable during the manufacture and use of the disposable apparatus, e.g., assembly 100. The machines (typically Spectra™ and Trima®, but which could include Prisma® and other Gambro or other manufacturers' blood processing machines) could then have a reader/writer connected thereto (either affixed thereto or therein in a fashion such as the reader/writer 174 shown in FIG. 4, or as a hand-held device such as the reader/writer 175 in FIG. 5). Standalone and/or hand-held readers and/or writers could also be used in manufacture (manufacturing step-by-step completion), sterilization completion, and at the customer site facilitating materials (e.g., tubing sets) handling; e.g., in inventory management (incoming deliveries and outgoing to use). The process may include placing coded data into the chip 15a which would identify materials (tubing sets) used, operator or other pertinent manufacturing data including, but not limited to, date of manufacture, sterilization completion (and/or type used, e.g. ethylene oxide (ETO) or gamma radiation).

The chip or chips or like devices could thus be written to at any or every pre-processing and/or manufacturing step also including, for example, but not limited to, the end-product actual size data (actual variance data, whether within acceptable ranges or not). The disposable assembly (e.g. tubing set 100) could then also be calibrated with information relating to actual pump stroke lengths (related to pump header actual size, length and inside diameter, e.g.), reservoir volumes and other important information which could help the processing machine (not shown) utilize the disposable assembly in a more effective way. For example, pump speeds may be altered in response to actual volume or pump header length information stored on a chip 15a. Once the disposable apparatus 100 with the chip 15a has left the manufacturing area the disposable apparatus can be tracked using RFID readers. Transportation information could also be added. This could facilitate easier inventory control for both the manufacturer and the end-user. The information can be read in a far field form from multiple apparatuses 100 serially or substantially simultaneously without needing to unload boxes or palettes for individual reading.

In use for and/or during processing, the chip 15a on the disposable apparatus 100 could impart information to the processing machine (not shown) such as expiration dates, type and calibration information. Disposable type information can affirm for or by the machine that the appropriate tubing and bag set is being disposed on/in the separation and collection machine for the corresponding procedure to be preformed thereby. Quality assurance may then be more automated thereby. Calibration information as previously mentioned could help tremendously in improving the end quality of the product delivered as well. During the processing ‘run,’ the processing machine could then impart information to the chip 15a, e.g., relating to machine ‘state’ and expected volume and valve positions. If power were to fail during a procedure, then a known recovery point could be understood and read back by a processing machine (the same machine or a different one) from process data stored on the chip 15a and the data relevant to the specific disposable apparatus 100 for later finishing of the process which could thus feasibly be finished on the same or a different processing machine. Field ‘failure’ returns of a disposable apparatus 100 would also be more valuable to the manufacturer since the chip 15a on the disposable apparatus 100 could contain and convey considerable information relating to the whole process, and thus provide explicit reasons for the failure which may then be designed around in future apparatuses. Thus the end-user could handle inventory, the manufacturer could manage field failures more effectively, and the processing machine could be tuned to the disposable apparatus more specifically to improve the efficiency of the blood component separation and/or collection process.

The pathogen inactivation agents which may be useful in this invention include any pathogen inactivation agents now or to be known to the art to be useful for inactivating pathogens or microorganisms. As such a pathogen inactivation agent may be a photosensitizer which is defined as any compound which absorbs radiation of one or more defined wavelengths and subsequently utilizes the absorbed energy to carry out a chemical process. Examples of such pathogen inactivation agents which may also be photosensitizers include porphyrins, psoralens, dyes such as neutral red, methylene blue, acridine, toluidines, flavine (acriflavine hydrochloride) and phenothiazine derivatives, coumarins, quinolones, quinones, and anthroquinones. Photosensitizers useful with this invention may include compounds which preferentially adsorb to nucleic acids, thus focusing their photodynamic effect upon pathogens, microorganisms and viruses with little or no effect upon accompanying cells or proteins. Other photosensitizers are also useful in this invention, such as those using singlet oxygen-dependent mechanisms. Most preferred are endogenous photosensitizers. The term endogenous means naturally found in a human or mammalian body, either as a result of synthesis by the body or because of ingestion as an essential foodstuff (e.g. vitamins) or formation of metabolites and/or byproducts in vivo. Examples of such endogenous photosensitizers are alloxazines such as 7,8-dimethyl-10-ribityl isoalloxazine (riboflavin), 7,8,10-trimethylisoalloxazine (lumiflavin), 7,8-dimethylalloxazine (lumichrome), isoalloxazine-adenine dinucleotide (flavine adenine dinucleotide [FAD]), alloxazine mononucleotide (also known as flavine mononucleotide [FMN] and riboflavine-5-phosphate), vitamin Ks, vitamin L, their metabolites and precursors, and napththoquinones, naphthalenes, naphthols and their derivatives having planar molecular conformations. The term “alloxazine” includes isoalloxazines. Endogenously-based derivative photosensitizers include synthetically derived analogs and homologs of endogenous photosensitizers which may have or lack lower (1-5) alkyl or halogen substituents of the photosensitizers from which they are derived, and which preserve the function and substantial non-toxicity thereof. When endogenous photosensitizers are used, particularly when such photosensitizers are not inherently toxic or do not yield toxic photoproducts after photoradiation, no removal or purification step is required after decontamination, and treated product can be directly returned to a patient's body or administered to a patient in need of its therapeutic effect.

Non-endogenous photosensitizers based on endogenous structures, such as those described in U.S. patent application Ser. No. 09/420,652, now U.S. Pat. No. 6,268,120, may also be included herein. These non-endogenous photosensitizers and endogenously-based derivative photosensitizers may be referred to herein as endogenously-based derivative photosensitizers.

Pathogens including microorganisms which may be eradicated using photosensitizers or other pathogen inactivation agents include, but are not limited to, viruses (both extracellular and intracellular), bacteria, bacteriophages, fungi, blood-transmitted parasites, and protozoa. Exemplary viruses include acquired immunodeficiency (HIV) virus, hepatitis A, B and C viruses, sinbis virus, cytomegalovirus, vesicular stomatitis virus, herpes simplex viruses, e.g. types I and II, human T-lymphotropic retroviruses, HTLV-III, lymphadenopathy virus LAV/IDAV, parvovirus, transfusion-transmitted (TT) virus, Epstein-Barr virus, and others known to the art. Bacteriophages include Φ X174, Φ 6, λ, R17, T₄, and T₂. Exemplary bacteria include P. aeruginosa, S. aureus, S. epidermis, L. monocytogenes, E. coli, K. pneumonia and S. marcescens.

The amount of pathogen inactivation agent or photosensitizer to be mixed with the fluid will be an amount sufficient to adequately inactivate pathogens or microorganisms therein, but less than a toxic (to humans or other mammals) or insoluble amount. Preferably the pathogen inactivation agent or photosensitizer is used in a concentration of at least about 1 μM up to the solubility of the pathogen inactivation agent or photosensitizer in the fluid, and preferably about 10 μM. For 7,8-dimethyl-10-ribityl isoalloxazine a concentration range between about 1 μM and about 160 μM is preferred, preferably about 10 μM. Irradiation may be at about 1 to about 200 J/cm² and/or may be made to take place for about 1 to about 60 minutes. Examples of methods and materials useful with the present invention are disclosed in U.S. Pat. No. 6,277,337 and No. 6,258,577, inter alia.

The fluid containing the photosensitizer may be exposed to pulsed or non-pulsed photoradiation of the appropriate wavelength and amount to activate the photosensitizer, but less than that which would cause non-specific damage to the biological components or substantially interfere with biological activity of other proteins present in the field. The wavelengths used may depend on the photosensitizer selected and the composition of the fluid, as is known in the art.

The above descriptions have been offered for illustrative purposes only, and are not intended to limit the scope of the invention of this application, which is as defined in the claims below.

1. A device for use in inactivating pathogens in a blood product comprising:
a container which is adapted to contain a blood product; and
an electromagnetically operable information chip which is connected to said container;
whereby the electromagnetically operable information chip is adapted for use in maintaining information about the blood product which may be contained within said container; and
whereby the information chip is adapted for the writing of information to the information chip including writing information to the information chip concerning the subjection of the container or the blood product to a pathogen inactivation process.

2. A device according to claim 1 whereby the information chip is also adapted for the reading of information therefrom.

3. A device according to claim 1 in which the electromagnetically operable information chip is adapted to be operable using radio frequency (RF) waves.

4. A device according to claim 2 in which said information chip is adapted to be operable with a discrete electromagnetic information chip reader.

5. A device according to claim 1 in which said information chip is adapted to be operable with a discrete electromagnetic information chip writer.

6. A device according to claim 1 in which said information chip is adapted to be operable with a discrete electromagnetic information chip reader/writer.

7. A device according to claim 1 in which said container has at least two side walls and a peripheral seam and said information chip is disposed on the seam of said container.

8. A device according to claim 1 in which said container has at least two sidewalls and a peripheral scam and wherein said information chip is attached to one of said sidewalls of said container.

9. A device according to claim 1 in which the information chip is adapted to have written thereto information to confirm the blood product contents of the container for pathogen inactivation.

10. A device according to claim 1 in which the information chip is adapted to have written thereto information about the type of pathogen inactivation procedure to which the container or the blood product will have been subjected.

11. A device according to claim 1 in which the information chip is adapted to have written thereto information about the pathogen inactivation agent to which the container or the blood product will have been subjected.

12. A device according to claim 1 in which the information chip is adapted to have written thereto information about the light irradiation pathogen inactivation procedure to which the container or the blood product will have been subjected.

13. A device according to claim 1 in which the container is adapted to be disposed in a pathogen inactivation housing which has defined therein an irradiation chamber so that the container and the blood product contents thereof may be irradiated in the irradiation chamber.

14. A device according to claim 13 in which at least one pin is positioned in said irradiation chamber, said pin being adapted to pass through at least one preformed aperture defined in said container so that said pin and said aperture coact to position said container so that the container and/or the blood product contents thereof will be properly irradiated and so that the information chip is positioned to be written to or read by an information chip reader or writer connected to the pathogen inactivation housing.

15. A device according to claim 13 in which said tray has a moveable bottom member such that when said tray is disposed in the irradiation chamber the bottom member is adapted to be moved to move the blood product contents of the container.

16. A device according to claim 1 in which said container is a first container which is adapted to have been connected to a second container in fluid communication for fluid transfer therebetween.

17. A device according to claim 16 in which the second container has a second electromagnetically operable information chip connected thereto for maintaining information about the second container or the fluid contents thereof.

18. A device according to claim 16 in which the second container contains a pathogen inactivation agent for exposure to the blood product contents of the first container.

19. A device according to claim 16 in which the second container is a blood product collection container from which a blood product may be transferred to the first container for the pathogen inactivation process.

20. A device for use in inactivating pathogens in a blood product comprising:
a container which is adapted to contain a blood product; and
an electromagnetically operable information chip which is connected to said container;
whereby the electromagnetically operable information chip is adapted for use in maintaining information about the blood product which may be contained within said container; and
whereby the information chip is adapted for the reading of information from the information chip including reading information from the information chip concerning the subjection of the container or the blood product to a pathogen inactivation process.

21. A method of inactivating pathogens in a blood product comprising:
disposing a blood product in a container, said container having connected thereto an electromagnetically operable information chip for use in maintaining information about the blood product contained within said container;
positioning the container so that the information chip is disposed to be written to by an information chip writer;
writing information to the information chip regarding the pathogen inactivation process; and
subjecting the blood product to a pathogen inactivation process.

22. A method according to claim 21 in which the subjecting step occurs before the positioning and writing steps.

23. A method according to claim 21 in which the subjecting step occurs substantially simultaneously with the positioning and writing steps.

24. A method according to claim 21 in which the electromagnetically operable information chip is adapted to be operable using radio frequency (RF) waves.

25. A method according to claim 21 in which said information chip is adapted to be operable with a discrete electromagnetic information chip reader.

26. A method according to claim 21 in which said information chip writer is also an information chip reader.

27. A method according to claim 21 in which the information chip is adapted to have written thereto information about the type of pathogen inactivation procedure to which the container or the blood product will have been subjected.

28. A method according to claim 21 in which the information chip is adapted to have written thereto information about the pathogen inactivation agent to which the container or the blood product will have been subjected.

29. A method according to claim 21 in which the information chip is adapted to have written thereto information about the light irradiation pathogen inactivation procedure to which the container or the blood product will have been subjected.

30. A method according to claim 21 wherein the container is made of a material that is substantially transparent to light irradiation.

31. A method according to claim 21 including placing the container into a pathogen inactivation housing which defines an inactivation chamber for the performance of the subjecting step.

32. A method according to claim 31 including the steps of placing said container in a tray having a bottom member, placing said tray in the inactivation chamber, and wherein the subjecting step includes irradiating said container or the blood product contents thereof in the inactivation chamber.

33. A method according to claim 31 in which said information chip is disposed on a seam of said container, said seam also defining at least a one preformed aperture for receiving a pin positioned in said chamber, the coaction of said pin with said aperture positioning said container for pathogen inactivation and so that the information chip is in a position to be written to and/or read by the information chip reader/writer.

34. A method according to claim 31 in which the information chip reader/writer is connected to the pathogen inactivation housing.

35. A method according to claim 21 in which the container has at least two walls which are at least substantially free of opaque indicia.

36. A method according to claim 21 in which said container has at least two sidewalls and a peripheral seam, and said information chip is disposed on the seam of said container.

37. A method according to claim 21 in which said container is a first container which is adapted to have been connected to a second container in fluid communication for fluid transfer therebetween.

38. A method according to claim 37 in which the second container has a second electromagnetically operable information chip connected thereto for maintaining information about the second container or the fluid contents thereof.

39. A method according to claim 37 in which the second container contains a pathogen inactivation agent for exposure to the blood product contents of the first container.

40. A method according to claim 37 in which the second container is a blood product collection container from which a blood product may be transferred to the first container for the pathogen inactivation process.

41. A system for processing a blood product comprising:
a blood processing device; and
a container having at least two side walls which is adapted to be disposed in operative relationship with the blood processing device, said container containing a blood product, the side walls of said container being made of a material which permits the desired processing to be effected on the blood product, said container having connected thereto an information chip for use in maintaining information about said container or the blood product contents thereof;
whereby said container is disposed in operative relationship to the blood processing device so that the information chip is in position to be written to and/or read by an information chip reader/writer; and
whereby information to be written to said information chip concerns the performance of the desired blood processing on the container or the blood product contents thereof.

42. A system according to claim 41 in which an identification chip reader is used to read the information from the information chip of said container to confirm that the container or the blood product contents thereof have been subjected to the desired procedure.

43. A system according to claim 41 in which the information chip is positioned on one of said side walls of said container.

44. A system according to claim 43 in which the information chip of said container is disposed on a seam of said container which is spaced from the contents of said container.

45. A system according to claim 41 in which the processing includes a pathogen inactivation procedure.

46. A system according to claim 41 in which the processing includes a light irradiation procedure.

47. A system according to claim 41 in which the processing includes a blood component separation procedure.

48. A system according to claim 41 in which the container is a blood cassette for use in a blood component separation procedure.

49. A system according to claim 41 in which the container is a blood cassette for use in a pathogen inactivation procedure.

50. A system according to claim 41 in which the container is a cuvette for use in a pathogen inactivation procedure.

51. A device for processing blood comprising:
a tubing set including a blood cassette, said cassette having connected thereto an information chip for use in maintaining information about the tubing set and/or blood cassette and/or blood product disposed therein;
whereby the information chip is adapted to have information written thereto concerning a processing of blood which will have been performed.

52. A device according to claim 51 in which the electromagnetically operable information chip is adapted to be operable using radio frequency (RF) waves.

53. A device according to claim 51 in which said information chip is adapted to be operable with a discrete electromagnetic information chip reader.

54. A device according to claim 51 in which said information chip is adapted to be operable with a discrete electromagnetic information chip writer.

55. A device according to claim 51 in which said information chip is adapted to be operable with a discrete electromagnetic information chip reader/writer.

56. A device according to claim 51 in which the processing includes a blood component separation procedure.

57. A device according to claim 51 in which the processing includes a pathogen inactivation procedure.

58. A device according to claim 51 in which the processing includes a light irradiation procedure.
